(19) United States
(12) Patent Application Publication
(10) Pub. No.: US 2011/0250251 A1
(43) Pub. Date: Oct. 13, 2011

(54) Cosmetic Methods And Compositions For Repairing Human Skin

(76) Inventors: Daniel H. Maes, Huntington, NY (US); Thomas Mammone, Farmingdale, NY (US); Kerri Goldgraben, Melville, NY (US)

(21) Appl. No.: 13/101,430

(22) Filed: May 5, 2011

Related U.S. Application Data
(63) Continuation of application No. 11/837,658, filed on Aug. 13, 2007, now abandoned.

(51) Int. Cl. A61K 8/66; A61Q 19/00; A61Q 19/08; A61K 8/11

(52) U.S. Cl. 424/401; 424/94.1; 424/94.61; 424/94.6

(57) ABSTRACT

The present invention relates to methods and compositions for repairing adverse effects of the environment, daily stress, sun exposure, or pre-mature aging on human skin, comprising applying to the skin, prior to a period of bodily rest, a topical composition that contains resveratrol or a derivative thereof and at least one DNA repair enzyme.

### CROSS REFERENCE

The present application is a continuation of U.S. Ser. No. 11/837,658, filed Aug. 13, 2007.

### FIELD OF THE INVENTION

The present invention relates to cosmetic methods and compositions for repairing adverse effects of the environment, daily stress, sun exposure, or pre-mature aging on human skin using resveratrol or resveratrol derivative and DNA repair enzymes.

### BACKGROUND OF THE INVENTION

The skin is made up of two major two major layers. The stratum corneum, or epidermis, is the top or outer layer of the skin. The primary function of the stratum corneum is to provide a protective covering and retard evaporative water loss from the aqueous interior. This is commonly referred to as the barrier function. The stratum corneum protects against mechanical insults, the ingress of foreign chemicals and assaults by microorganisms. It also provides the first defense against ultraviolet light, screening out more than 80% of incident ultraviolet B irradiation.

The dermis lies under the epidermis and makes up 90 percent of the skin's thickness. The dermis contains a dense meshwork of collagen and elastin, providing strength and elasticity to the skin. Fibroblasts constitute the main cell type present in the dermis. Fibroblasts are responsible for synthesis and secretion of dermal matrix components, including collagen, elastin, and glycosaminoglycans (such as hyaluronic acid). Whereas collagen provides strength to the skin and elastin its elasticity, glycosaminoglycans serve to keep the skin moist and plump.

Free oxygen radicals, harsh chemicals, sun exposure, daily stress, and other environmental factors may have adverse effects on human skin. For example, UV radiation can damage DNA molecules in the skin cells by cross-linking adjacent pyrimidines on the same DNA strand and forming pyrimidine dimers. The cells have developed several different cellular mechanisms for repairing and removing the DNA damages. However, when the DNA damages were incurred too fast, e.g., by high intensity UV radiation, the DNA repair system may become overloaded. Consequently, un-repaired DNA damages start to accumulate and, if reaching certain threshold, may lead to pre-mature skin aging or even cancerous development in the skin.

In view of the many adverse effects impacting the skin, there is a need for cosmetic compositions and methods that can effectively ameliorate such adverse effects on the skin, rejuvenate the skin, and improve the appearance and condition of the skin.

### SUMMARY OF THE INVENTION

In one aspect, the present invention relates to a method for repairing or ameliorating the adverse effects of the environment, daily stress, sun exposure, or pre-mature aging on human skin, comprising applying to the skin, prior to a period of bodily rest, a composition comprising resveratrol or a derivative thereof and at least one DNA repair enzyme.

In another aspect, the present invention relates to a method for treating skin subjected to adverse effects of the environment, daily stress, sun exposure, or pre-mature aging, comprising sequentially treating the skin with at least two different compositions, in any order, wherein the first composition comprises resveratrol or a derivative thereof and at least one DNA repair enzyme, and wherein the second composition comprises at least one active ingredient that improves the efficacy of the first composition when both compositions are applied to the skin.

In a further aspect, the present invention relates to a skin care kit for repairing or ameliorating the adverse effects of the environment, daily stress, sun exposure, or pre-mature aging on human skin, comprising a first receptacle containing a first composition comprising resveratrol or a derivative thereof and at least one DNA repair enzyme, and a second receptacle containing a second composition comprising at least one active ingredient that improves the efficacy of the first composition in the first receptacle when both compositions are applied to the skin.

In yet another aspect, the present invention relates to a topical composition for repairing or ameliorating the adverse effects of the environment, daily stress, sun exposure, or pre-mature aging on human skin, comprising resveratrol or a derivative thereof and 8-oxoguanine DNA glycosylate in a pharmaceutically or cosmetically acceptable vehicle.

Other aspects and objectives of the present invention will become more apparent from the ensuing description, examples, and claims.

### DETAILED DESCRIPTION OF THE INVENTION, AND PREFERRED EMBODIMENTS THEREOF

The present invention provides methods and compositions for repairing adverse effects of the environment, daily stress, sun exposure, or pre-mature aging on human skin. The term “repairing the adverse effects . . . on human skin” is used herein to designate arresting, reversing, ameliorating, diminishing, and/or reducing defects, imperfections, or aesthetically unpleasant conditions of the skin, which include, but are not limited to: age spots, sunburn, sun spots, lines, fine lines, wrinkles, crow's feet, spider veins, stretch marks, dark eye circles, hyperpigmentation, hypopigmentation, discoloration, uneven skin tone, dullness, freckles, skin breakout, blemishes, skin fragility, dryness, patchiness, tactile roughness, chapping, sagginess, thinning, enlarged pores, cellulite formation, acne formation, rosacea, psoriasis, and eczema. The term “skin” includes facial or body skin as well as lips.

Specifically, the composition of the invention comprises resveratrol or a derivative thereof and at least one DNA repair enzyme and methods for treating skin with this composition. It is believed that the resveratrol or resveratrol derivative and the DNA repair enzyme in such a topical composition act in synergy to boost or enhance the natural repair responses in the skin cells and therefore improve the effectiveness of cellular repair mechanism against adverse effects of the environment, daily stress, sun exposure, or pre-mature aging on human skin. It is also believed that when an individual is resting, the skin of such an individual is more receptive to active ingredients that will help restore and revitalize its appearance, and the natural repair responses in the skin cells can be most effectively boosted or enhanced. Correspondingly, it is desirable to apply the topical composition of the present invention to the skin prior to a period of bodily rest, which can be either a nightly sleep (e.g., from about 3 to about 10 hours) or a nap (e.g., from about 15 minutes to about 4 hours).

The compositions of the invention are further described as follows.

### 1. DNA Repair Enzyme

The composition of the invention contains at least one DNA repair enzyme. The term “DNA repair enzyme” refers to enzymes now known or subsequently discovered or developed, including glycosylases, apurinic/apyrimidinic endonucleases or other enzymes having activities capable of repairing damaged DNA. Suitable DNA repair enzymes for use in the present invention may include, but are not limited to: 8-oxoguanine DNA glycosylase, uracil-and-hypoxanthine-DNA-glycosylase, damaged-base glycosylase (e.g., 3-methyl-ladenine-DNA glycosylase), 3-methyladenine-DNA-glycosylase, pyrimidine dimer-specific glycosylase, pyrimidine glycosylase/abasic lyase, N-glycosylase/apyrimidinic lyase, N-glycosylase/apurinic-apyrimidinic lyase, photolyase, O⁶-methylguanine-DNA-methyl transferase, T4 endonuclease V, pyrimidine dimer-specific endonuclease, apyrimidin/apurin-endonuclease, UV damage endonuclease, correndonuclease, and DNA exonuclease. Other DNA repair enzymes or enzyme complexes involved in either the base excision repair (BER) pathway, the nucleotide excision repair (NER) pathway, or the alternative excision repair pathway can also be used for practice of the present invention. Such DNA repair enzymes may be derived or extracted from suitable sources, such as bacteria, algae, protozoans, planktons, plants, and the like.

Preferably, but not necessarily, the DNA repair enzymes are encapsulated in liposomes, either alone or in combination with resveratrol or resveratrol derivative and/or one or more additional skin care actives. Liposomes are microscopic vesicles consisting of an aqueous core enclosed in one or more lipid layers formed by membrane lipids, such as phospholipids and sphingomyelins. Liposomes facilitate transfer of cosmetically active agents, such as the DNA repair enzyme, resveratrol or a derivative thereof, and other skin care actives, into the dermis of skin. For more details regarding encapsulation of DNA repair enzymes in liposomes, see U.S. Pat. No. 5,296,231, the contents of which are incorporated herein by reference in their entireties for all purposes.

For example, 8-oxoguanine DNA glucosylase (OGG1), which is a DNA repair enzyme derived from the plant Arabidopsis thaliana that repairs the oxidative 8-oxoguanine damages in both genomic and mitochondrial DNA, is commercially available in a liposomal formulation containing lecithin and water under the tradename ROXISOMES™ from AGI Dermatics at Freeport, N.Y. For another example, T4 endonuclease V (T4N5), which is a DNA repair enzyme derived from Micrococcus luteus cell lysate that repairs UVB-induced cyclobutane pyrimidine dimers (CPD), is also commercially available in a liposomal formulation under the tradename ULTRASOMES™ or ULTRASOMES-V™ from AGI Dermatics. Further, photolyase, which is a DNA repair enzyme derived from ocean plankton that repair pyrimidine dimers upon activation of visible light, is commercially available in a liposomal formulation under the tradename PHOTOSOMES™ from AGI Dermatics. The above-described exemplary liposome encapsulated DNA repair enzymes can be provided in the topical composition of the present invention in an amount ranging from about 0.01% to 20%, preferably from about 0.1% to about 10%, and more preferably desirably from about 0.5% to about 2%, by total weight of the total composition.

### II. Resveratrol or Derivatives Thereof

The composition of the invention further contains resveratrol or a derivative thereof. Resveratrol, also referred to as 3,5,4′-trihydroxystilbene, is a polyhydroxy-substituted stilbene compound present in red grapes, raspberries, blueberries, and certain other plant berries or extracts, which has the general formula:

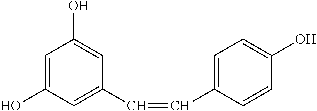

Resveratrol has been shown to be an effective antioxidant and also exhibits strong anti-proliferative and anti-inflammatory properties. It has recently been reported that resveratrol can mimic caloric restriction (CR) in various organisms, such as yeast, roundworms, fruit-flies, short-lived fish, and mice, slow the aging process in such organisms, and significantly extend their life spans. Although not wishing to be bound by any specific theory, inventors of the present invention believe that resveratrol can reduce cell proliferation and slow down the apoptosis process, thereby allowing more time for DNA damage repair in the cells. It is postulated that resveratrol, when combined with a DNA repair enzyme, can result in a synergistic effect on boosting or otherwise enhancing the natural DNA repair capacity of the cells.

However, resveratrol may be potentially unstable in certain cosmetic formulations. Specifically, resveratrol is susceptible to hydrolysis in aqueous-based formulations and may cause such formulations to become discolored. One way to address the instability of resveratrol in aqueous-based formulations is to modify the resveratrol by substituting the hydroxy groups at the 3, 5, and 4′ position with other functional groups to form resveratrol derivatives that are more stable in cosmetic formulas. It has been discovered that resveratrol derivatives of inorganic acids, organic carboxylic acids, mono-, di-, or polysaccharides, or other functional groups are more stable in aqueous-based formulations. The substitutional groups not only function to protect and stabilize the phenol groups of resveratrol and make the resveratrol derivative more suitable for use in aqueous-based cosmetic formulations, but they can also be easily hydrolyzed from the compound upon application to the skin, preferably by enzymes and other ingredients on the skin surface, to release an active form of resveratrol into the skin. The resveratrol derivatives of the present invention have a general formula of:

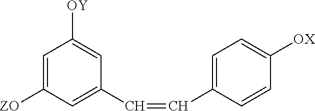

wherein X, Y, and Z are either hydrogen or a protective group, provided that at least one of X, Y, and Z is the protective group. Exemplary resveratrol derivatives suitable for use in the cosmetic or topical compositions of the present invention are described in greater detail hereinafter.

A. Resveratrol Esters of Inorganic or Organic Acids

Resveratrol esters of inorganic acids, in which one or more of the X, Y, and Z are inorganic acid functional groups such as phosphates, nitrates, sulfonates, and carbonates, can be used in the present invention. Following is a list of exemplary inorganic acid esters that are particularly suitable for practice of the present invention:

| | |
|---|---|
| 3-phosphate-5,4′-dihydroxystilbene | 5-phosphate-3,4′-dihydroxystilbene |
| 4′-phosphate-3,5-dihydroxystilbene | 3,5-diphsophate-4′-hydroxystilbene |
| 3,4′-diphosphate-5-hydroxystilbene | 4′,5-diphosphate-3-hydroxystilbene |
| 3,5,4′-triphosphate stilbene | 3-nitrate-5,4′-dihydroxystilbene |
| 5-nitrate-3,4′-dihydroxystilbene | 4′-nitrate-3,5-dihydroxystilbene |
| 3,5-dinitrate-4′-hydroxystilbene | 3,4′-dinitrate-5-hydroxystilbene |
| 4′,5-dinitrate-3-hydroxystilbene | 3,5,4′-trinitrate stilbene |
| 3-sulfonate-5,4′-dihydroxystilbene | 5-sulfonate-3,4′-dihydroxystilbene |
| 4′-sulfonate-3,5-dihydroxystilbene | 3,5-disulfonate-4′-hydroxystilbene |
| 3,4′-disulfonate-5-hydroxystilbene | 4′,5-disulfonate-3-hydroxystilbene |
| 3,5,4′-trisulfonate stilbene | 3-carbonate-5,4′-dihydroxystilbene |
| 5-carbonate-3,4′-dihydroxystilbene | 4′-carbonate-3,5-dihydroxystilbene |
| 3,5-dicarbonate-4′-hydroxystilbene | 3,4′-dicarbonate-5-hydroxystilbene |
| 4′,5-dicarbonate-3-hydroxystilbene | 3,5,4′-tricarbonate stilbene. |

Pharmaceutically acceptable salts of the above-listed resveratrol esters can also be used in the cosmetic compositions of the present invention. Such salts may include one or more monovalent or divalent cations selected from the group consisting of Na, K, Mg, Ca, Fe, and NH₄. The salts can be formed by adding corresponding bases, such as sodium hydroxide, potassium hydroxide, and the like, into a solution containing the resveratrol esters.

The inorganic acid esters of resveratrol may be readily formed by well known chemical processes that substitute the hydroxyl groups of phenols or polyphenols with the phosphate, sulfonates, and carbonate functional groups. For example, U.S. Pat. No. 4,003,966 describes a one-step process for selectively phosphorylating phenols to form phosphate esters thereof, the contents of which are hereby incorporated herein by reference in their entireties for all purposes.

A particularly preferred resveratrol derivative for practice of the present invention is the 3, 4′, 5-triphosphate stilbene, also referred to as a resveratrol triphosphate ester having the formula of:

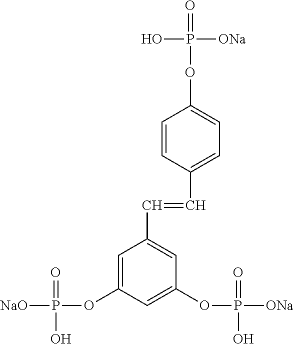

Phosphate esters of resveratrol, including resveratrol triphosphate, are disclosed in International Patent Application Publication No. WO 2006/029484A1, which is hereby incorporated by reference in its entirety. Resveratrol triphosphate may be synthesized by the method as set forth in Example 2 of WO 2006/029484A1. More specifically, a solution of resveratrol (3,4,5-trihydroxystilbene) (25 mmols, 5.7 grams) and dimethylaminopyridine (7.5 mmols, 0.93 grams) in 100 ml acetonitrile is cooled under nitrogen up to −10° C. After 10 minutes, carbon tetrachloride (375 mmol, 36.2 ml) and DIEA (159 mmol; 27.7 ml) and the mixture maintained under stirring for 30 minutes. Dibenzylphosphate (113 mmols, 25.0 ml) is added and the mixture stirred for an additional 12 hours at room temperature. The course of the reaction is monitored by TLC (silica F254, eluent ethyl acetate/n-hexane 80/20 v/v). One liter of 0.5 M KH2PO4 is added, and the mixture then extract with ethyl acetate. The resulting product, tri(dibenzylphosphate) resveratrol, is purified by filtration on a silica gel, washing first with a mixture of ethyl acetate/n-hexane (80/20 v/v) to remove any remaining unreacted resveratrol, and then with methanol, to obtain a yellow oil.

To the tri(dibenzylphosphate) resveratrol (12.5 mmol) in 200 mL of anhydrous DCM at 0° C., is added bromomethylsilane (79 mmols, 10.4 mL). After 2 hours, 300 mL of H2O is added, and the reaction mixture is stirred for 1 hour. The water phase is washed again with ethyl acetate, then lyophilized to obtain an orange oil.

To the product obtained above, solubilized in 400 mL of ethanol, is added CH3Na (37 mmol; 2.03 g) and the reaction stirred for 12 hours at room temperature. The ethanol is evaporated in a rotavapor, and the residue solubilized in H2O. The water phase is washed with ethyl acetate and lyophilized. The mass spectrum of the resulting white solid shows the presence of resveratrol triphosphate (PM=468.1), with a total yield of >90% with respect to resveratrol.

If desired, the resveratrol triphosphate may be neutralized with organic or inorganic bases such as sodium hydroxide, potassium hydroxide and the like. Particularly preferred is where the resveratrol triphosphate is neutralized with sodium hydroxide to form trisodium resveratrol triphosphate. Resveratrol triphosphate may also be purchased from Ajinomoto in the neutralized form, having the CTFA name trisodium resveratrol triphosphate.

B. Carboxylic Acid Esters of Resveratrol

Another group of resveratrol derivatives that can be used in the present invention is esters of resveratrol and aliphatic or aromatic carboxylic acids, in which one or more of X, Y, and Z is a —C(O)—R₁ group, wherein Rₜ is selected from the group consisting of linear, branched, saturated or unsaturated, or cyclic C₁-C₄₀ alkyl, substituted C₁-C₄₀ alkyl, C₁-C₄₀ alkenyl, substituted C₁-C₄₀ alkenyl, C₁-C₄₀ alkynyl, substituted C₁-C₄₀ alkynyl, aryl, C₁-C₄₀ aryl, and C₁-C₄₀ substituted aryl. In one preferred embodiment, the R group is a straight or branched chain fatty, or C₆₋₃₀, saturated or unsaturated alkyl group. The substituents may be selected from C₁-C₄₀ straight or branched chain, saturated or unsaturated alkyl, halogen (such as fluoro), hydrogen, alkoxy, hydroxyl, and the like.

Exemplary carboxylic acids that can be used to form ester of resveratrol include, but are not limited to: saturated monocarboxylic acids, such as acetic acid, propionic acid, butyric acid (C4), valeric acid, hexanoic acid, caprylic acid (C8), lauric acid, stearic acid (C18), isostearic acid (branched C18), linoleic acid, Iinolenic acid, myristic acid (C 14), arachidic acid (C20), arichidonic acid, erucic acid, behenic acid (C22), lauric acid (C12), capric acid (C10), caproic (C6), and palmitic acid (C16); unsaturated monocarboxylic acids, such as acrylic acid, methacrylic acid, sorbic acid, oleic acid, linoleic acid, linolenic acid, docosahexaenoic acid, and eicosapentaenoic acid; amino acids, such as arginine, glutamine, and tyrosine; keto acids, such as pyruvic acid and acetoacetic acid; aromatic carboxylic acids, such as ascorbic acid, benzoic acid, salicylic acid, and ferulic acid; di- and tri-carboxylic acids, such as oxalic acid, malonic acid, malic acid, succinic acid, and glutaric acid. The designation “C” followed by a number indicates the number of carbon atoms in the alkyl chain.

Following is a list of exemplary carboxylic acid esters of resveratrol that are particularly suitable for practice of the present invention:

| | |
|---|---|
| 3-acetate-5,4′-dihydroxystilbene | 5-acetate-3,4′-dihydroxystilbene |
| 4′-acetate-3,5-dihydroxystilbene | 3,5-diacetate-4′-hydroxystilbene |
| 3,4′-diacetate-5-hydroxystilbene | 4′,5-diacetate-3-hydroxystilbene |
| 3,5,4′-triacetate stilbene | 3-propionate-5,4′-dihydroxystilbene |
| 5-propionate-3,4′-dihydroxystilbene | 4′-propionate-3,5-dihydroxystilbene |
| 3,5-dipropionate-4′-hydroxystilbene | 3,4′-dipropionate-5-hydroxystilbene |
| 4′,5-dipropionate-3-hydroxystilbene | 3,5,4′-tripropionate stilbene |
| 3-butyrate-5,4′-dihydroxystilbene | 5-butyrate-3,4′-dihydroxystilbene |
| 4′-butyrate-3,5-dihydroxystilbene | 3,5-dibutyrate-4′-hydroxystilbene |
| 3,4′-dibutyrate-5-hydroxystilbene | 4′,5-dibutyrate-3-hydroxystilbene |
| 3,5,4′-tributyrate stilbene | 3-valerate-5,4′-dihydroxystilbene |
| 5-valerate-3,4′-dihydroxystilbene | 4′-valerate-3,5-dihydroxystilbene |
| 3,5-divalerate-4′-hydroxystilbene | 3,4′-divalerate-5-hydroxystilbene |
| 4′,5-divalerate-3-hydroxystilbene | 3,5,4′-trivalerate stilbene |
| 3-hexanoate-5,4′-dihydroxystilbene | 5-hexanoate-3,4′-dihydroxystilbene |
| 4′-hexanoate-3,5-dihydroxystilbene | 3,5-dihexanoate-4′-hydroxystilbene |
| 3,4′-dihexanoate-5-hydroxystilbene | 4′,5-dihexanoate-3-hydroxystilbene |
| 3,5,4′-trihexanoate stilbene | 3-caprylate-5,4′-dihydroxystilbene |
| 5-caprylate-3,4′-dihydroxystilbene | 4′-caprylate-3,5-dihydroxystilbene |
| 3,5-dicaprylate-4′-hydroxystilbene | 3,4′-dicaprylate-5-hydroxystilbene |
| 4′,5-dicaprylate-3-hydroxystilbene | 3,5,4′-tricaprylate stilbene |
| 3-laurate-5,4′-dihydroxystilbene | 5-laurate-3,4′-dihydroxystilbene |
| 4′-laurate-3,5-dihydroxystilbene | 3,5-dilaurate-4′-hydroxystilbene |
| 3,4′-dilaurate-5-hydroxystilbene | 4′,5-dilaurate-3-hydroxystilbene |
| 3,5,4′-trilaurate stilbene | 3-stearate-5,4′-dihydroxystilbene |
| 5-stearate-3,4′-dihydroxystilbene | 4′-stearate-3,5-dihydroxystilbene |
| 3,5-distearate-4′-hydroxystilbene | 3,4′-distearate-5-hydroxystilbene |
| 4′,5-distearate-3-hydroxystilbene | 3,5,4′-tristearate stilbene |
| 3-palmitate-5,4′-dihydroxystilbene | 5-palmitate-3,4′-dihydroxystilbene |
| 4′-palmitate-3,5-dihydroxystilbene | 3,5-dipalmitate-4′-hydroxystilbene |
| 3,4′-dipalmitate-5-hydroxystilbene | 4′,5-dipalmitate-3-hydroxystilbene |
| 3,5,4′-tripalmitate stilbene | 3-acrylate-5,4′-dihydroxystilbene |
| 5-acrylate-3,4′-dihydroxystilbene | 4′-acrylate-3,5-dihydroxystilbene |
| 3,5-diacrylate-4′-hydroxystilbene | 3,4′-diacrylate-5-hydroxystilbene |
| 4′,5-diacrylate-3-hydroxystilbene | 3,5,4′-triacrylate stilbene |
| 3-methacrylate-5,4′-dihydroxystilbene | 5-methacrylate-3,4′-dihydroxystilbene |
| 4′-methacrylate-3,5-dihydroxystilbene | 3,5-dimethacrylate-4′-hydroxystilbene |
| 3,4′-dimethacrylate-5-hydroxystilbene | 4′,5-dimethacrylate-3-hydroxystilbene |
| 3,5,4′-trimethacrylate stilbene | 3-sorbate-5,4′-dihydroxystilbene |
| 5-sorbate-3,4′-dihydroxystilbene | 4′-sorbate-3,5-dihydroxystilbene |
| 3,5-disorbate-4′-hydroxystilbene | 3,4′-disorbate-5-hydroxystilbene |
| 4′,5-disorbate-3-hydroxystilbene | 3,5,4′-trisorbate stilbene |
| 3-oleate-5,4′-dihydroxystilbene | 5-oleate-3,4′-dihydroxystilbene |
| 4′-oleate-3,5-dihydroxystilbene | 3,5-dioleate-4′-hydroxystilbene |
| 3,4′-dioleate-5-hydroxystilbene | 4′,5-dioleate-3-hydroxystilbene |
| 3,5,4′-trioleate stilbene | 3-linoleate-5,4′-dihydroxystilbene |
| 5-linoleate-3,4′-dihydroxystilbene | 4′-linoleate-3,5-dihydroxystilbene |
| 3,5-dilinoleate-4′-hydroxystilbene | 3,4′-dilinoleate-5-hydroxystilbene |
| 4′,5-dilinoleate-3-hydroxystilbene | 3,5,4′-trilinoleate stilbene |
| 3-linolenate-5,4′-dihydroxystilbene | 5-linolenate-3,4′-dihydroxystilbene |
| 4′-linolenate-3,5-dihydroxystilbene | 3,5-dilinolenate-4′-hydroxystilbene |
| 3,4′-dilinolenate-5-hydroxystilbene | 4′,5-dilinolenate-3-hydroxystilbene |
| 3,5,4′-trilinolenate stilbene | 3-docosahexaenoate-5,4′-dihydroxystilbene |
| 5-docosahexaenoate-3,4′-dihydroxystilbene | 4′-docosahexaenoate-3,5-dihydroxystilbene |
| 3,5-didocosahexaenoate-4′-hydroxystilbene | 3,4′-didocosahexaenoate-5-hydroxystilbene |
| 4′,5-didocosahexaenoate-3-hydroxystilbene | 3,5,4′-tridocosahexaenoate stilbene |
| 3-eicosapentaenoic-5,4′-dihydroxystilbene | 5-eicosapentaenoic-3,4′-dihydroxystilbene |
| 4′-eicosapentaenoic-3,5-dihydroxystilbene | 3,5-dieicosapentaenoic-4′-hydroxystilbene |
| 3,4′-dieicosapentaenoic-5-hydroxystilbene | 4′,5-dieicosapentaenoic-3-hydroxystilbene |
| 3,5,4′-trieicosapentaenoic stilbene | 3-arginate-5,4′-dihydroxystilbene |
| 5-arginate-3,4′-dihydroxystilbene | 4′-arginate-3,5-dihydroxystilbene |
| 3,5-diarginate-4′-hydroxystilbene | 3,4′-diarginate-5-hydroxystilbene |
| 4′,5-diarginate-3-hydroxystilbene | 3,5,4′-triarginate stilbene |
| 3-glutamate-5,4′-dihydroxystilbene | 5-glutamate-3,4′-dihydroxystilbene |
| 4′-glutamate-3,5-dihydroxystilbene | 3,5-diglutamate-4′-hydroxystilbene |
| 3,4′-diglutamate-5-hydroxystilbene | 4′,5-diglutamate-3-hydroxystilbene |
| 3,5,4′-triglutamate stilbene | 3-tyrosate-5,4′-dihydroxystilbene |
| 5-tyrosate-3,4′-dihydroxystilbene | 4′-tyrosate-3,5-dihydroxystilbene |
| 3,5-dityrosate-4′-hydroxystilbene | 3,4′-dityrosate-5-hydroxystilbene |
| 4′,5-dityrosate-3-hydroxystilbene | 3,5,4′-trityrosate stilbene |
| 3-pyruvate-5,4′-dihydroxystilbene | 5-pyruvate-3,4′-dihydroxystilbene |
| 4′-pyruvate-3,5-dihydroxystilbene | 3,5-dipyruvate-4′-hydroxystilbene |
| 3,4′-dipyruvate-5-hydroxystilbene | 4′,5-dipyruvate-3-hydroxystilbene |
| 3,5,4′-tripyruvate stilbene | 3-acetoacetate-5,4′-dihydroxystilbene |
| 5-acetoacetate-3,4′-dihydroxystilbene | 4′-acetoacetate-3,5-dihydroxystilbene |
| 3,5-diacetoacetate-4′-hydroxystilbene | 3,4′-diacetoacetate-5-hydroxystilbene |
| 4′,5-diacetoacetate-3-hydroxystilbene | 3,5,4′-triacetoacetate stilbene |
| 3-ascorbate-5,4′-dihydroxystilbene | 5-ascorbate-3,4′-dihydroxystilbene |
| 4′-ascorbate-3,5-dihydroxystilbene | 3,5-diascorbate-4′-hydroxystilbene |
| 3,4′-diascorbate-5-hydroxystilbene | 4′,5-diascorbate-3-hydroxystilbene |
| 3,5,4′-triascorbate stilbene | 3-benzoate-5,4′-dihydroxystilbene |
| 5-benzoate-3,4′-dihydroxystilbene | 4′-benzoate-3,5-dihydroxystilbene |
| 3,5-dibenzoate-4′-hydroxystilbene | 3,4′-dibenzoate-5-hydroxystilbene |
| 4′,5-dibenzoate-3-hydroxystilbene | 3,5,4′-tribenzoate stilbene |
| 3-salicylate-5,4′-dihydroxystilbene | 5-salicylate-3,4′-dihydroxystilbene |
| 4′-salicylate-3,5-dihydroxystilbene | 3,5-disalicylate-4′-hydroxystilbene |
| 3,4′-disalicylate-5-hydroxystilbene | 4′,5-disalicylate-3-hydroxystilbene |
| 3,5,4′-trisalicylate stilbene | 3-ferulate-5,4′-dihydroxystilbene |
| 5-ferulate-3,4′-dihydroxystilbene | 4′-ferulate-3,5-dihydroxystilbene |
| 3,5-diferulate-4′-hydroxystilbene | 3,4′-diferulate-5-hydroxystilbene |
| 4′,5-diferulate-3-hydroxystilbene | 3,5,4′-triferulate stilbene |
| 3-oxalate-5,4′-dihydroxystilbene | 5-oxalate-3,4′-dihydroxystilbene |
| 4′-oxalate-3,5-dihydroxystilbene | 3,5-dioxalate-4′-hydroxystilbene |
| 3,4′-dioxalate-5-hydroxystilbene | 4′,5-dioxalate-3-hydroxystilbene |
| 3,5,4′-trioxalate stilbene | 3-malonate-5,4′-dihydroxystilbene |
| 5-malonate-3,4′-dihydroxystilbene | 4′-malonate-3,5-dihydroxystilbene |
| 3,5-dimalonate-4′-hydroxystilbene | 3,4′-dimalonate-5-hydroxystilbene |
| 4′,5-dimalonate-3-hydroxystilbene | 3,5,4′-trimalonate stilbene |
| 3-malate-5,4′-dihydroxystilbene | 5-malate-3,4′-dihydroxystilbene |
| 4′-malate-3,5-dihydroxystilbene | 3,5-dimalate-4′-hydroxystilbene |
| 3,4′-dimalate-5-hydroxystilbene | 4′,5-dimalate-3-hydroxystilbene |
| 3,5,4′-trimalate stilbene | 3-succinate-5,4′-dihydroxystilbene |
| 5-succinate-3,4′-dihydroxystilbene | 4′-succinate-3,5-dihydroxystilbene |
| 3,5-disuccinate-4′-hydroxystilbene | 3,4′-disuccinate-5-hydroxystilbene |
| 4′,5-disuccinate-3-hydroxystilbene | 3,5,4′-trisuccinate stilbene |
| 3-glutarate-5,4′-dihydroxystilbene | 5-glutarate-3,4′-dihydroxystilbene |
| 4′-glutarate-3,5-dihydroxystilbene | 3,5-diglutarate-4′-hydroxystilbene |
| 3,4′-diglutarate-5-hydroxystilbene | 4′,5-diglutarate-3-hydroxystilbene |
| 3,5,4′-triglutarate stilbene | 3-glutarate-5,4′-dihydroxystilbene |
| 5-glutarate-3,4′-dihydroxystilbene | 4′-glutarate-3,5-dihydroxystilbene |
| 3,5-diglutarate-4′-hydroxystilbene | 3,4′-diglutarate-5-hydroxystilbene |
| 4′,5-diglutarate-3-hydroxystilbene | 3,5,4′-triglutarate stilbene. |

One particularly preferred group of carboxylic acid esters of resveratrol are either saturated or unsaturated fatty acid esters of resveratrol, such as resveratrol butyrates, resveratrol valerates, resveratrol hexanoates, resveratrol sorbates, resveratrol laurates, resveratrol stearates, resveratrol palmitates, resveratrol oleates, resveratrol linoleates, resveratrol linolenates, resveratrol eicosapentaenoates, and resveratrol docosahexanoates. Such fatty acid esters of resveratrol can be readily formed by esterification of resveratrol with acid derivatives according to the Schotten-Baumann reaction in alkaline aqueous medium, as described by U.S. Pat. No. 6,572,882, the content of which is incorporated herein by reference in its entirety for all purposes.

Another particularly preferred group of carboxylic acid esters of resveratrol are the aromatic carboxylic acid esters of resveratrol, such as resveratrol ferulates, which can be formed by reacting resveratrol with ferulic acid in aqueous medium.

C. Resveratrol Ether Derivatives

Yet another group of resveratrol derivatives that can be used in the present invention are resveratrol ethers, in which one or more of X, Y, and Z is —R₂, wherein R₂ is selected from the group consisting of linear, branched or cyclic C₁-C₄₀ alkyl, substituted C₁-C₄₀ alkyl, C₁-C₄₀ alkenyl, substituted C₁-C₄₀ alkenyl, C₁-C₄₀ alkynyl, substituted C₁-C₄₀ alkynyl, C₁-C₄₀ aryl, substituted C₁-C₄₀ aryl, and mono-, di-, oligo-, and polysaccharides. Following is a list of exemplary resveratrol ethers that are particularly suitable for practice of the present invention:

| | |
|---|---|
| 3-methoxy-5,4′-dihydroxystilbene | 5-methoxy-3,4′-dihydroxystilbene |
| 4′-methoxy-3,5-dihydroxystilbene | 3,5-dimethoxy-4′-hydroxystilbene |
| 3,4′-dimethoxy-5-hydroxystilbene | 4′,5-dimethoxy-3-hydroxystilbene |
| 3,5,4′-trimethoxy stilbene | 3-ethoxy-5,4′-dihydroxystilbene |
| 5-ethoxy-3,4′-dihydroxystilbene | 4′-ethoxy-3,5-dihydroxystilbene |
| 3,5-diethoxy-4′-hydroxystilbene | 3,4′-diethoxy-5-hydroxystilbene |
| 4′,5-diethoxy-3-hydroxystilbene | 3,5,4′-triethoxy stilbene |
| 3-propyloxy-5,4′-dihydroxystilbene | 5-propyloxy-3,4′-dihydroxystilbene |
| 4′-propyloxy-3,5-dihydroxystilbene | 3,5-dipropyloxy-4′-hydroxystilbene |
| 3,4′-dipropyloxy-5-hydroxystilbene | 4′,5-dipropyloxy-3-hydroxystilbene |
| 3,5,4′-tripropyloxy stilbene | 3-phenyloxy-5,4′-dihydroxystilbene |
| 5-phenyloxy-3,4′-dihydroxystilbene | 4′-phenyloxy-3,5-dihydroxystilbene |
| 3,5-diphenyloxy-4′-hydroxystilbene | 3,4′-diphenyloxy-5-hydroxystilbene |
| 4′,5-diphenyloxy-3-hydroxystilbene | 3,5,4′-triphenyloxy stilbene |
| 3-glucoside-5,4′-dihydroxystilbene | 5-glucoside-3,4′-dihydroxystilbene |
| 4′-glucoside-3,5-dihydroxystilbene | 3,5-diglucoside-4′-hydroxystilbene |
| 3,4′-diglucoside-5-hydroxystilbene | 4′,5-diglucoside-3-hydroxystilbene |
| 3,5,4′-triglucoside stilbene. | |

In one specific embodiment of the present invention, a methoxy-substituted resveratrol derivative is used. For example, the compositions of the present invention may comprise 3,5-dimethoxy-4′-hydroxystilbene, which can be extracted from the Indian Kino Tree (Pterocarpus marsupium) and is commercially available under the trade name PTEROSTILBENE from Sigma-Aldrich at St. Louis, Mo.

In another specific embodiment of the present invention, the resveratrol derivative contains one or more saccharide-containing protective groups, such as glucose, galactose, mannose, fructose, sucrose, lactose, maltose, trehalose, and the like. For example, resveratrol glucoside, which can be obtained by extraction from plants or plant material such as polygonum cuspidatum tissue or in vitro cultures of vitis vinifera cells, is used in the cosmetic compositions of the present invention.

D. Nitrogen-Containing Derivatives of Resveratrol

The resveratrol derivatives used in the compositions of the present invention may also contain one or more nitrogen-containing functional groups, i.e., one or more of OX, OY and OZ in the above formula, on page 6, are selected from the group consisting of amides, amines, imines, amidines, and carboxamidines. The following is a list of exemplary nitrogen-containing derivatives of resveratrol that are particularly suitable for practice of the present invention:

| | |
|---|---|
| 3-amide-5,4′-dihydroxystilbene | 5-amide-3,4′-dihydroxystilbene |
| 4′-amide-3,5-dihydroxystilbene | 3,5-diamide-4′-hydroxystilbene |
| 3,4′-diamide-5-hydroxystilbene | 4′,5-diamide-3-hydroxystilbene |
| 3,5,4′-triamide stilbene | 3-amino-5,4′-dihydroxystilbene |
| 5-amino-3,4′-dihydroxystilbene | 4′-amino-3,5-dihydroxystilbene |
| 3,5-diamino-4′-hydroxystilbene | 3,4′-diamino-5-hydroxystilbene |
| 4′,5-diamino-3-hydroxystilbene | 3,5,4′-triamino stilbene |
| 3-imino-5,4′-dihydroxystilbene | 5-imino-3,4′-dihydroxystilbene |
| 4′-imino-3,5-dihydroxystilbene | 3,5-diimino-4′-hydroxystilbene |
| 3,4′-diimino-5-hydroxystilbene | 4′,5-diimino-3-hydroxystilbene |
| 3,5,4′-triimino stilbene | 3-amidino-5,4′-dihydroxystilbene |
| 5-amidino-3,4′-dihydroxystilbene | 4′-amidino-3,5-dihydroxystilbene |
| 3,5-diamidino-4′-hydroxystilbene | 3,4′-diamidino-5-hydroxystilbene |
| 4′,5-diamidino-3-hydroxystilbene | 3,5,4′-triamidino stilbene. |

Preferably, but not necessarily, the resveratrol derivatives of the present invention are encapsulated in liposomes, either alone or in combination with the DNA repair enzyme and/or one or more additional skin care actives, for more effective delivery thereof into the dermis of skin. The resveratrol derivatives may be present in the cosmetic composition of the present invention at an amount ranging from about 0.001% to about 95%, preferably from about 0.005% to about 90%, more preferably from about 0.1% to about 20%, by total weight of the total composition.

### III. Additional Ingredients

The compositions of the invention may contain one or more additional ingredients for further improving the efficacy of resveratrol or resveratrol derivative and the DNA repair enzyme combination in repairing the adverse effects of the environment, daily stress, sun exposure, or pre-mature aging on human skin, or improving the aesthetics and stability of the compositions containing the resveratrol or resveratrol derivative and DNA repair enzyme so that such compositions are commercially acceptable. Such additional ingredients may include, but are not limited to: oils, surfactants, humectants, botanical extracts, vitamins, antioxidants, sunscreen agents, preservatives, and the like. The composition may be in the form of an emulsion, gel, suspension, aqueous solution, or in the anhydrous form. If present in the form of an emulsion, the composition may be in the form of a water-in-oil or oil-in-water emulsion. Suggested ranges of water are from about 0.1 to 99%, preferably from about 1-85%, more preferably from about 5 to 80% by weight of the total composition, and suggested ranges of oil from about 1-85%, preferably from about 3-80%, more preferably from about 5-75% by weight of the total composition. If the composition is present in the anhydrous form, it may also contain one or more oils, and if so, suggested ranges are from about 1 to 95% by weight of the total composition.

Suitable oils include materials also known as skin conditioning agents such as nonvolatile silicones, esters, paraffinic hydrocarbons, vegetable oils, and synthetic oils. The term “nonvolatile” as used herein means that the compound has a vapor pressure of less than about 2 mm of mercury at 20° C. Preferably, the skin conditioning agent is characterized by a viscosity from about 5 to 10 centistokes at 25° C. up to about 1,000,000 centipoise at 25° C. Particularly preferred are the nonvolatile silicones, including but not limited to: amine functional silicones such as amodimethicone, phenyl substituted silicones such as bisphenylhexamethicone, trimethylsiloxyphenyl dimethicone, phenyl trimethicone, polyphenylmethylsiloxane, dimethicone, phenyl dimethicone, diphenyl dimethicone, and dimethicone substituted with C₂₋₃₀ alkyl groups such as cetyl dimethicone. Suitable esters include mono-, di-, or triesters. Monoesters are in the general form RCO—R′ wherein R and R′ are each independently a C₁₋₄₅ straight or branched chain, saturated or unsaturated alkyl. Diesters may be formed by the reaction of a C₁₋₄₅ aliphatic or aromatic mono- or dihydric alcohol with a C₁₋₄₅ aliphatic or aromatic mono- or dicarboxylic acid, as appropriate, where the aliphatic group may be straight or branched chain, or saturated or unsaturated. Suitable triesters include the reaction products of a C₁₋₄₅ aliphatic or aromatic alcohol having at least three hydroxyl groups with a C₁₋₄₅ carboxylic acid, or a C₁₋₄₅ aliphatic or aromatic alcohols with a C₁₋₄₅ tricarboxylic acid, with the aliphatic chains being linear or branched, saturated or unsaturated. Examples include esters of caprylic and capric acids and glycerin such as caprylic/capric triglycerides; esters of glycerin or polyglycerin and stearic acid such as glyceryl stearate, diglyceryl diisostearate; esters of malic acid and isostaryl alcohol such as diisostearyl malate; coco caprylate caprate and the like.

Humectants which may be used in the compositions of the invention and include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C₁₋₆, preferably C₂₋₄ alkylene glycols, most particularly butylene glycol. If present, such humectants may range from about 0.001% to about 25%, preferably from about 0.005% to about 20%, more preferably from about 0.1% to about 15%, by total weight of the topical composition.

Suitable botanical extracts that may be used in the compositions of the invention include extracts from plants (herbs, roots, flowers, fruits, seeds) such as flowers, fruits, vegetables, and so on, including yeast ferment extract, padica pavonica extract, thermus thermophilis ferment extract, camelina sativa seed oil, boswellia serrata extract, olive extract, aribodopsis thaliana extract, acacia dealbata extract, acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and those set forth on pages 1646 through 1660 of the CTFA Cosmetic Ingredient Handbook, Eighth Edition, Volume 2. Further specific examples include, but are not limited to, Glycyrrhiza Glabra, Salix Nigra, Macrocycstis Pyrifera, Pyrus Malus, Saxifraga Sarmentosa, Vilis Vinifera, Morus Nigra, Scutellaria Baicalensis, Anthemis Nobilis, Salvia Sclarea, Rosmarinus Officianalis, Citrus Medica Limonum, Panax Ginseng, and mixtures thereof. If presented, the amount of botanical extracts preferably ranges from about 0.0001% to about 10%, preferably about 0.0005% to about 8%, more preferably about 0.001% to about 5%, by total weight of the topical composition.

Sunscreen agents that can be used in the compositions of the present invention include, but are not limited to: benzophenones and derivatives thereof (e.g., benzophenone-3, dioxybenzone, sulisobenzone, octabenzone, hydroxy- and/or methoxy-substituted benzophenones, and benzophenonesulfonic acids and salts thereof); salicylic acid derivatives (e.g., ethylene glycol salicylate, triethanolamine salicylate, octyl salicylate, homomenthyl salicylate, and phenyl salicylate); urocanic acid and derivatives thereof (e.g., ethyl urocanate); p-aminobenzoic acid (PABA) and derivatives thereof (e.g., ethyl/isobutyl/glyceryl esters thereof and 2-ethylhexyl p-dimethylaminobenzoate, which is also referred to as octyldimethyl PABA); anthranilates and derivatives thereof (e.g., o-amino-benzoates and various esters of amino-benzoic acid); benzalmalonate derivatives; benzimidazole derivatives; imidazolines; bis-benzazolyl derivatives; dibenzoylmethanes and derivatives thereof (e.g., 4-tert-butyl-4′-methoxydibenzoylmethane, which is commonly referred to as “avobenzone,” and 4-isopropyl-dibenzoyl methane); benzoxazole, benzodiazole, benzotriazoles, and derivatives thereof (e.g., 2-(2-hydroxy-5-methylphenyl) benzotriazole and methylene bis-benzotriazolyl tetramethylbutylphenol, which is commonly referred to as “Tinosorb M”); diphenylacrylates and derivatives thereof (e.g., 2-ethylhexyl-2-cyano-3,3-diphenylacrylate, which is commonly referred to as “octocrylene,” and ethyl-2-cyano-3,3-diphenylacrylate, which is commonly referred to as “etocrylene”); diesters or polyesters containing diphenylmethylene or 9H-fluorene substitutional groups; 2-phenyl-benzimidazole-5-sulphonic acid (PBSA); 4,4-diarylbutadienes; cinnamates and derivatives thereof (e.g., 2-ethylhexyl-p-methoxycinnamate, octyl-p-methoxycinnamate, umbelliferone, methylumbelliferone, methylaceto-umbelliferone, esculetin, methylesculetin, and daphnetin); camphors and derivatives thereof (e.g., 3-benzylidenecamphor, 4-methylbenzylidenecamphor, polyacrylamidomethyl benzylidenecamphor, benzylidene camphor sulfonic acid, and terephthalylidene dicamphor sulfonic acid); triazines and derivatives thereof (e.g., 2,4-bis-{[4-(2-ethyl-hexyloxy)-2-hydroxyl]-phenyl}-6-(4-methoxyphenyl)-1,3,5-triazine, which is commonly referred to as “Tinosorb S”); naphthalates and derivatives thereof (e.g., diethylhexyl-2,6-naphthalate); naphtholsulfonates and derivatives thereof (e.g., sodium salts of 2-naphthol-3,6-disulfonic and 2-naphthol-6,8-disulfonic acids); dibenzalacetone and benzalacetonephenone; diphenylbutadienes and derivatives thereof; di-hydroxynaphthoic acid and salts thereof; o- and p-hydroxybiphenyldisulfonates; coumarin derivatives (e.g., 7-hydroxy, 7-methyl, and 3-phenyl derivatives thereof); azoles/diazoles/triazoles and derivatives thereof (e.g., 2-acetyl-3-bromoindazole, phenyl benzoxazole, methyl naphthoxazole, and various aryl benzotriazoles); quinine and derivatives thereof (e.g., bisulfate, sulfate, chloride, oleate, and tannate salts thereof); quinoline and derivatives thereof (e.g., 2-phenylquinoline and 8-hydroxyquinoline salts); tannic acid and derivatives thereof (e.g., hexaethylether derivatives thereof); hydroquinone and derivatives thereof; uric acid and derivatives thereof; vilouric acid and derivatives thereof, and mixtures or combinations thereof. Salts and otherwise neutralized forms of certain acidic sunscreens from the list hereinabove are also useful herein. Particularly preferred sunscreen agents for the present invention are: 4,4′-t-butylmethoxy-dibenzoylmethane, 2-ethylhexyl-2-cyano-3,3-diphenylacrylate, 2-ethylhexylsalicylate, 3,3,5-trimethylcyclohexylsalicylate, 2-ethylhexyl p-methoxycinnamate, 2-hydroxy-4-methoxybenzophenone, 2,2-dihydroxy-4-methoxybenzophenone, 2,4-bis-{4-(2-ethyl-hexyloxy)-2-hydroxyl]-phenyl}-6-(4-methoxyphenyl)-1,3,5-triazine, methylene bis-benzotriazolyl tetramethylbutylphenol, terephthalylidene dicamphor sulfonic acid, diethylhexyl 2,6-naphthalate, digalloyltrioleate, ethyl 4-[bis(hydroxypropyl)]aminobenzoate, glycerol p-aminobenzoate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl-sulfoniobenzoxazoic acid, and mixtures or combinations thereof. The above-described sunscreen agents may be used alone or in combination of two or more. In addition, other known animal or vegetable extracts having ultraviolet light-absorbing ability may properly be used alone or in combination. If presented, the amount of sunscreen agents preferably ranges from about 0.001% to about 50%, preferably about 0.01% to about 10%, more preferably about 1% to about 5%, by total weight of the topical composition.

The compositions of the present invention may further contain vitamins and/or antioxidants. Suitable vitamins may include ascorbic acid and derivatives thereof, such as ascrobyl palmitate; the 13 vitamins such as thiamine, riboflavin, pyridoxin, and the like; Vitamin A and the ester-based derivatives thereof, such as palmitate, acetate, and the like, as well as Vitamin A in the form of beta carotene; Vitamin E and derivatives thereof, such as Vitamin E acetate, nicotinate, or other esters thereof; Vitamins D and K; coenzymes such as thiamine pyrophoshate, flavin adenin dinucleotide, folic acid, pyridoxal phosphate, tetrahydrofolic acid, and the like. Suitable antioxidants may include potassium sulfite, sodium bisulfite, sodium erythrobate, sodium metabisulfite, sodium sulfite, propyl gallate, cysteine hydrochloride, butylated hydroxytoluene, butylated hydroxyanisole, and so on. If present, the amount of vitamins and/or antioxidants may each range from about 0.001% to about 10%, preferably from about 0.01% to about 8%, more preferably from about 0.05% to about 5%, by total weight of the topical composition.

The composition may also contain one or more surfactants, particularly if present in the emulsion form. Preferably such surfactants are nonionic and may be in the form of silicones or organic nonionic surfactants. Suggested ranges are from about 0.1 to 40%, preferably from about 0.5 to 35%, more preferably from about 1 to 30% by weight of the total composition. Suitable silicone surfactants include polyorganosiloxane polymers that have amphiphilic properties, for example contain hydrophilic radicals and lipophilic radicals. These silicone surfactants may be liquids or solids at room temperature. Exemplary silicone surfactants that can be used in the present invention include, but are not limited to: dimethicone copolyols, alkyl dimethicone copolyols, and emulsifying silicone elastomers. Emulsifying silicone elastomers are elastomers that have one or more hydrophilic groups such as hydroxyl, oxyethylene, and the like bonded thereto so as to confer hydrophilic properties to the elastomer. Suitable organic nonionic surfactants may include alkoxylated alcohols or ethers formed by the reaction of an alcohol with a polyalkyleneoxide containing repeating units of alkylene oxide. Preferably, the alcohol is a fatty alcohol having 6 to 30 carbon atoms. Examples of organic nonionic surfactants that can be used in the present invention include, but are not limited to: steareth 2-100, beheneth 5-30, ceteareth 2-100, ceteth 1-45, and the like, which are formed by polyethyleneoxide with the corresponding stearyl/behenyl/cetyl alcohol (wherein the number as used herein designates the number of repeating units of ethylene oxide in the polyethyleneoxide). Other alkoxylated alcohols include esters formed by reaction of polymeric alkylene glycols with glyceryl fatty acid, such as PEG glyceryl oleates, PEG glyceryl stearate; or PEG polyhydroxyalkanotes such as PEG dipolyhydroxystearate wherein the number of repeating ethylene glycol units ranges from 3 to 1000. Also suitable as nonionic surfactants are formed by the reaction of a carboxylic acid with an alkylene oxide or with a polymeric ether. Monomeric, homopolymeric, or block copolymeric ethers, alkoxylated sorbitan, alkoxylated sorbitan derivatives can also be used as nonionic surfactants in the present invention.

The compositions of the invention may also contain other ingredients such as structuring agents in the form of polymeric structuring agents such as acrylic polymers, polyamides or polyurethanes. The structuring agents may be water or oil soluble or dispersible. Such structuring agents will provide structure, or increase the viscosity of the composition. If present, suggested ranges are from about 0.1 to 50%, preferably from about 0.5 to 40%, more preferably from about 1 to 35% by weight of the total composition. Suitable structuring agents include natural, synthetic waxes, or mineral waxes such as petrolatum, candelilla, ozokerite, synthetic wax, polyethylene, and so on. Suitable polymeric structuring agents include acrylic polymers such as carbopol or pemulen (polymers of acrylic acid, methacrylic acid, or their simple esters crosslinked by polyfunctional agents such as ally ethers of sucrose or pentaerythritol), ester or amide terminated polyamides such as those sold by Arizona Chemical under the Uniclear or Sylvaclear trademarks, or aqueous dispersions or solutions of polyurethanes.

In the case where the compositions of the invention are colored, from about 0.1 to 80%, more preferably from about 0.5 to 75%, more preferably from about 1 to 70% by weight of the total composition of particulates may be present. The term “particulates” refers to pigments in the form of inorganic or organic pigments such as iron oxides (black, blue, red, yellow), or the D&C and FD&C Lakes. Particulates may also include ingredients commonly referred to as “powders” that is particulate materials that are present for muting color (such as titanium dioxide) or providing bulk to the composition. Further examples include nylon, polymethylmethacrylate, silica, silica silylate, and the like.

### IV. Forms of the Cosmetic or Topical Compositions

The ingredients as described hereinabove are preferably provided in a cosmetic compositions that may be formulated into a cream, gel, lotion, oil, ointment, powder, stick, cake, or other forms that can be topically applied. The resulting cosmetic or topical composition may be in the form of a liquid, solid, semi-solid, dispersion, suspension, solution or emulsion, and it can be either aqueous-based or anhydrous. The compositions of the invention may also be in the form of color cosmetic compositions, such as foundation makeup, mascara, lip color, blush, eye shadow, and the like. Particularly, it is preferable to formulate the resveratrol derivative into either the water phase or the oil phase of an emulsion, depending on the type of derivative. For example, certain hydrophilic derivatives such as resveratrol triphosphate, resveratrol trisulfonate, and the like are water soluble and will generally be found in the water phase of the emulsion. Certain other derivatives are lipophilic in nature and will more likely be found in the oil phase of the emulsion. The DNA repair enzyme is preferably found in the water phase of the emulsion or encapsulated in an aqueous phase within liposomes.

Typical skin creams or lotions comprise from about 5-98% water, 1-85% oil, and from about 0.1 to 20% of one or more surfactants.

Typical color cosmetic compositions such as foundations, blush, eye shadow and the like may be in the anhydrous or aqueous form. If aqueous based, such compositions will preferably contain from about 5-98% water, 1-85% oil, and optionally from about 0.1 to 20% of one or more surfactants in addition to from about 0.1 to 65% of particulates that are pigments or a combination of pigments and powders. If anhydrous, the compositions may contain from about 0.1 to 95% oil, from about 0.1 to 99% particulates, and optionally from about 0.1 to 50% of one or more structuring agents.

Typical mascara compositions generally contain from about 5-98% water, 1-85% oil, and from about 0.1 to 20% surfactant in addition to natural or synthetic polymers that are film forming, such as aqueous dispersions of acrylic copolymers, aqueous dispersions of polyurethane, or silicone resins.

Typical lip color compositions are in the form of sticks or glosses, and generally comprise from about 0.1 to 95% oil, from about 0.1 to 60% structuring agent, and from about 0.1 to 50% particulates.

Typical toner compositions comprise from about 0.1 to 99% of water or other polar nonaqueous solvent such as ethanol, propylene glycol, butylene glycol. Toners are typically applied for cleansing purposes using a cotton pad or other applicator to swipe across the skin to remove debris or dirt.

Typical spritzer compositions include those that may be sprayed on the skin. Preferably such compositions will contain from about 0.1 to 99% of water or other polar nonaqueous solvent. Such compositions are generally applied as leave on compositions.

Typical gels are aqueous based and may contain from about 0.1 to 95% water, from about 0.1 to 50% structuring agents.

### V. Regimens and Kits

In the case where the composition is applied to the skin in a single formula, the composition may be in any of the forms as described above. In addition, the skin may be treated in a regimen involving application of two or more compositions sequentially. In such case the composition containing the DNA repair enzyme and the resveratrol or a derivative thereof will be a separate composition which may be in any one of the forms mentioned above. In such case a second composition in any one of the forms above is then applied to the skin and generally will contain ingredients that enhance the efficacy, stability, or aesthetics of the first applied composition.

For example, a composition containing the DNA repair enzyme and resveratrol or a derivative thereof may be applied to the skin first in the form of a toner, spritzer, or cleanser, facial treatment mask and the like. Thereafter a skin cream, lotion, or the like containing the ingredients mentioned above may be applied as a leave on composition. In another example, the skin may be treated with a skin cream or lotion followed by treatment of the skin with the composition containing the DNA repair enzyme and the resveratrol or a derivative thereof. The skin cream or lotion may be first applied followed by a foundation makeup composition containing the resveratrol or a derivative thereof and DNA repair enzyme. In yet another example, the skin may be treated with a facial treatment mask followed by application of a skin cream or lotion containing the resveratrol or a derivative thereof and the DNA repair enzyme.

The invention also includes a skin treatment kit containing at least one receptacle containing the composition with the resveratrol or a derivative thereof and DNA repaid enzyme and a second receptacle containing a skin cream or lotion that contains ingredients that enhance the effectiveness of the other applied composition.

In yet another embodiment, the resveratrol or resveratrol derivative may be found in one composition and the DNA repair enzyme in another composition, with the compositions being in any one or more of the forms mentioned above, and the compositions are sequentially applied.

### VI. Methods of Application

The methods of application in the present invention will depend on the ultimate intended use of composition. The cosmetic or topical compositions can be applied locally to the area particularly susceptible to adverse effects of the environment, daily stress, sun exposure, or pre-mature aging, such as face, throat, and hand, or it can be applied to the entire body of the user.

The cosmetic or topical compositions of the present invention may be applied to the skin on an as-needed basis, or according to a pre-set schedule. The topical or cosmetic compositions of the present invention may be applied directly to clean skin, before application of any moisturizer, foundation, make-up, etc. Alternatively, such compositions can be applied over moisturizer, and optionally over foundation and/or make-up. The amount applied each time, the area of application, the duration of application, and the frequency of application can vary widely, depending on the specific need of the user. For example, the cosmetic or topical compositions can be applied for a period of days to months or even years, and at a frequency ranging from about once per week to about five times per day. For another example, the compositions can be applied for a period of about six months and at a frequency ranging from about three times a week to about three times per day, and preferably about once or twice per day. In one embodiment, the composition of the invention is applied on a daily basis prior to sleep as part of a permanent skin care regimen. Specifically, the face is washed, and the composition is applied to skin immediately prior to bedtime.

As mentioned hereinabove, it is believed that when an individual is resting, the skin of such an individual is more receptive to active ingredients that will help restore and revitalize its appearance, and the natural repair responses in the skin cells can be most effectively boosted or enhanced during such resting. Correspondingly, it is particularly desirable to apply the cosmetic or topical composition of the present invention to the skin prior to a period of bodily rest. For example, the cosmetic or topical composition can be applied from about 1 to 60 minutes before a nightly sleep, which may last, for example, from about 4 to about 10 hours. Alternatively, the cosmetic or topical composition can be applied shortly before a nap or meditation, which may last, for example, from about 15 minutes to about 4 hours.

In one specific embodiment of the present invention, the cosmetic or topical composition of the present invention can be formulated as a night cream or a night repair serum, which can be applied to the face of an individual before sleep without rinsing off. In another specific embodiment of the present invention, the cosmetic or topical composition of the present invention is formulated as an overnight facial mask, which can be applied to the face before sleep, left thereon overnight, and then rinsed off the next morning. The cosmetic or topical composition of the present invention can also be formulated as a regular facial mask or peel, which can be applied to the face of the individual for a relatively short period of time, for example, from about 3 minutes to about 1 hour, while the individual is allowed to take a nap or meditate (e.g., during a spa session in combination with aromatherapy or massage), and then rinsed off or otherwise removed at the end of such period.

The invention will be further described in connection with the following examples which are set forth for the purposes of illustration only.

### EXAMPLES

### Example 1

### Demonstration of the Synergistic Effects of OGG1 Enzyme and Resveratrol in Protecting Cells from UVB Induced Toxicity

In this test, human keratinocyte cells were challenged with UVB, following treatments with ROXISOMES™ (liposome encapsulated OGG1 enzyme from AG1 Dermatics) alone, resveratrol alone, or the combination of ROXISOMES™ and resveratrol. The keratinocytes survival rates were then compared to see the effects of different treatments on the kertinocytes viability.

Specifically, normal human keratinocytes were cultured in Epilife Medium with Human Keratinocyte Growth Supplement. The cells were sub-cultured into 96-well plates. A first set of plates were treated overnight with resveratrol at testing concentrations of 0 (which was used as the control or base measurement), 1, 5, and 25 μM, respectively. A second set of plates were treated with ROXISOMES™ at 0% (which was used as the control or base measurement), 0.04%, 0.2%, and 1%, respectively. A third set of plates were treated with a combination of resveratrol and ROXISOMES™ at 0%/0 μM (which was used as the control or base measurement), 0.04%/1 μM, 0.2%/5 μM, and 1%/25 μM, respectively. The keratinocytes were then subjected to UVB irradiation (in PBS buffer) at doses of 0, 20, 40, 60, 80, or 100 mJ/cm². After aspiration of the PBS buffer, the cells were post-treated with the same concentrations of actives. The cells were assayed for viability utilizing MTS reagent (from CellTiter96, Promega). Absorbance was read at 490 nm, following an approximately two hour incubation at 37° C./5% CO₂.

The following tables show the percentage of increase in survival rate for cells treated with the actives at medium and high concentrations over the survival rates of cells in the control plates that were not treated with any actives under the same dosage of UVB radiation:

TABLE 1

| UVB Radiation (mJ/cm²) | 0 | 20 | 40 | 60 | 80 | 100 |
|---|---|---|---|---|---|---|
| Roxisomes (0.2%) | 37% | 19% | 17% | 13% | 16% | 24% |
| Resveratrol (5 μM) | 11% | 12% | 14% | 11% | 16% | 16% |
| Combination (0.2%/5 μM) | 24% | 22% | −3% | 40% | 52% | 60% |

TABLE 2

| UVB Radiation (mJ/cm²) | 0 | 20 | 40 | 60 | 80 | 100 |
|---|---|---|---|---|---|---|
| Roxisomes (1%) | 42% | 18% | 20% | 6% | 31% | 42% |
| Resveratrol (25 μM) | −6% | −7% | −9% | −3% | 0% | 9% |
| Combination (1%/25 μM) | −14% | −12% | −26% | 20% | 36% | 65% |

The above tables show that when the intensity of UVB radiation reaches a sufficiently high level, for example, more than 60 mJ/cm², cells treated with the combination of resveratrol and OGG1 enzyme achieve a synergistic increase in the survival rate over the control cells, i.e., the increase of the combination is greater than the sum of increases achieved separately by resveratrol and OGG1 enzyme.

It is important to note that when the intensity of UVB radiation is relatively low, the cell survival rate is influenced by various factors, and DNA damages caused by the UVB radiation have relatively less impact on the cell survival rate in relation to other factors. Therefore, the protection provided by improved DNA repair process shows less influence on the cell survival rate. However, when the intensity of UVB radiation reaches a sufficiently high level, DNA damages due to the UVB radiation become a major cause of cell death and have relatively more impact on the cell survival rate in relation to other factors, and improved DNA repair process therefore exhibits more significant influence on the cell survival rate at higher UVB intensities.

### Example 2

### Demonstration of the Synergistic Effects of T4N5 Enzyme and Resveratrol in Protecting Cells from UVB induced Toxicity

In this test, human keratinocyte cells were challenged with UVB, following treatments with T4 endonuclease V (T4N5) enzyme alone, resveratrol alone, or the combination of T4N5 enzyme and resveratrol. The keratinocytes survival rates were then compared to see the effects of different treatments on the kertinocytes viability.

Specifically, normal human keratinocytes were cultured in Epilife Medium with Human Keratinocyte Growth Supplement. The cells were sub-cultured into 96-well plates. A first set of plates were treated with resveratrol at testing concentrations of 0 (which was used as the control or base measurement), 1, 5, and 25 μM, respectively. A second set of plates were treated with T4N5 enzyme at 0% (which was used as the control or base measurement), 0.04%, 0.2%, and 1%, respectively. A third set of plates were treated with a combination of resveratrol and T4N5 enzyme at 0%/0 μM, 0.04%/1 μM, 0.2%/5 μM, and 1%/25 μM, respectively. The keratinocytes were then subjected to UVB irradiation (in PBS buffer) at doses of 0, 20, 40, 60, 80, or 100 mJ/cm². After aspiration of the PBS buffer, the cells were post-treated with the same concentrations of actives. The cells were assayed for viability utilizing MTS reagent (from CellTiter96, Promega). Absorbance was read at 490 nm on the SpectraMax spectrophotometer (from Molecular Devices), following an approximately two hour incubation at 37° C./5% CO₂.

The following tables show the percentage of increase in survival rate for cells treated with the actives at medium and high concentrations over the survival rates of cells in the control plates that were not treated with any actives:

TABLE 3

| UVB Radiation (mJ/cm²) | 0 | 20 | 40 | 60 | 80 | 100 |
|---|---|---|---|---|---|---|
| T4N5 (0.2%) | 17% | 21% | 30% | 42% | 57% | 50% |
| Resveratrol (5 μM) | 17% | 24% | 28% | 12% | −11% | −11% |
| Combination (0.2%/5 μM) | 32% | 42% | 56% | 87% | 131% | 161% |

TABLE 4

| UVB Radiation (mJ/cm²) | 0 | 20 | 40 | 60 | 80 | 100 |
|---|---|---|---|---|---|---|
| T4N5 (1%) | −7% | 2% | 9% | 17% | 47% | 51% |
| Resveratrol (25 μM) | 4% | 10% | 14% | 4% | 0 | 9% |
| Combination (1%/25 μM) | 14% | 22% | 47% | 69% | 125% | 162% |

The above tables show that at higher UVB intensities, e.g., 60, 80, or 100 mJ/cm², cells treated with the combination of resveratrol and T4N5 enzyme achieve a synergistic increase in the survival rate over the control cells, i.e., the increase of the combination is greater than the sum of increases achieved separately by resveratrol and the T4N5 enzyme.

### Example 3

### Demonstration of the Synergistic Effects of T4N5 Enzyme and Resveratrol Triphosphate in Protecting Cells from UVB Induced Toxicity

In this test, human keratinocyte cells were challenged with UVB, following treatments with T4 endonuclease V (T4N5) enzyme alone, resveratrol triphosphate alone, or the combination of T4N5 enzyme and resveratrol triphosphate. The keratinocytes survival rates were then compared to see the effects of different treatments on the kertinocytes viability.

Specifically, normal human keratinocytes were cultured in Epilife Medium with Human Keratinocyte Growth Supplement. The cells were sub-cultured into 96-well plates. A first set of plates were treated with resveratrol triphosphate at testing concentrations of 0 (which was used as the control or base measurement), 1, 5, and 25 μM, respectively. A second set of plates were treated with T4N5 enzyme at 0% (which was used as the control or base measurement), 0.04%, 0.2%, and 1%, respectively. A third set of plates were treated with a combination of resveratrol triphosphate and T4N5 enzyme at 0%/0 μM, 0.04%/1 μM, 0.2%/5 μM, and 1%/25 μM, respectively. The keratinocytes were then subjected to UVB irradiation (in PBS buffer) at doses of 0, 40, 80, 120, 160, or 200 mJ/cm². After aspiration of the PBS buffer, the cells were post-treated with the same concentrations of actives. The cells were assayed for viability utilizing MTS reagent (from CellTiter96, Promega). Absorbance was read at 490 nm on the SpectraMax spectrophotometer (from Molecular Devices), following an approximately two hour incubation at 37° C./5% CO₂.

The following tables show the percentage of increase in survival rate for cells treated with the actives at medium and high concentrations over the survival rates of cells in the control plates that were not treated with any actives:

TABLE 5

| UVB Radiation (mJ/cm²) | 0 | 40 | 80 | 120 | 160 | 200 |
|---|---|---|---|---|---|---|
| T4N5 (0.2%) | −5% | −11% | 4% | −2% | −6% | −7% |
| Resveratrol Triphosphate | −17% | −17% | −12% | −1% | 30% | 52% |
| (5 μM) | | | | | | |
| Combination (0.2%/5 μM) | −6% | −18% | −16% | −6% | 29% | 60% |

TABLE 6

| UVB Radiation (mJ/cm²) | 0 | 40 | 80 | 120 | 160 | 200 |
|---|---|---|---|---|---|---|
| T4N5 (1%) | 0 | −1% | 15% | −5% | −2% | −8% |
| Resveratrol Triphosphate | −2% | −2% | 0% | 18% | 58% | 68% |
| (25 μM) | | | | | | |
| Combination (1%/25 μM) | −2% | −1% | 5% | 24% | 69% | 62% |

The above tables show that at higher UVB intensities, e.g., 160 and 200 mJ/cm², cells treated with the combination of resveratrol triphosphate and T4N5 enzyme achieve a synergistic increase in the survival rate over the control cells, i.e., the increase of the combination is greater than the sum of increases achieved separately by resveratrol triphosphate and the T4N5 enzyme.

### Example 4

### Formulations

FORMULA 1

| Component | Wt % |
|---|---|
| Stearic acid | 2.40 |
| Glyceryl monostearate | 2.20 |
| Butyl paraben | 0.10 |
| Mineral oil/Lanolin alcohol | 9.50 |
| Petrolatum/Lanolin alcohol | 2.00 |
| Sesame oil | 4.30 |
| Propyl paraben | 4.30 |
| Deionized water | QS |
| Triethanolamine 99% | 0.82 |
| Methyl paraben | 0.30 |
| Trisodium EDTA | 0.10 |
| Propylene glycol | 4.30 |
| Sodium ribonucleic acid | 0.10 |
| Trisodium resveratrol triphosphate | 0.50 |
| ROXISOMES ™ (water/Arabidopsis thaliana extract/lecithin) | 0.10 |
| ULTRASOMES-V ™ (water/lecithin/Micrococcus lysate) | 0.20 |

FORMULA 2

| Component | Wt % |
|---|---|
| Deionized water | QS |
| Hydroxyethyl urea | 0.50 |
| Hyaluronic acid | 9.00 |
| Disodium EDTA | 0.12 |
| Creatine | 0.05 |
| Sucrose | 0.50 |
| Caffeine | 0.20 |
| Caprylyl glycol | 0.40 |
| Caprylic/capric triglyceride/cetyl alcohol/ | 4.00 |
| C12-20 acid PEG-8 ester | |
| PEG-100 stearate | 1.20 |
| C12-20 acid PEG-8 ester | 4.96 |
| Caprylic/capric triglyceride | 0.55 |
| Behenyl alcohol | 0.50 |
| Coco caprylate caprate | 5.10 |
| Sweet almond oil | 0.10 |
| Dimethicone, 100 cst. | 2.50 |
| Sesame oil | 0.10 |
| Potassium cetyl phosphate | 0.50 |
| Apricot kernel oil | 0.10 |
| Wheat bran extract/olive extract | 0.20 |
| Cholesterol | 0.20 |
| Linoleic acid | 0.20 |
| Cholesterol/potassium sulfate | 0.20 |
| Theobroma grandiflorum seed butter | 1.40 |
| Lauryl PCA | 0.01 |
| Dimethicone | 1.50 |
| Phenoxyethanol | 0.70 |
| Water/polyaminopropyl biguanide | 0.40 |
| Glycerin | 2.00 |
| Butylene glycol | 1.00 |
| Mica/titanium dioxide | 1.00 |
| Pearl powder | 0.001 |
| Silica | 0.50 |
| 30% aqueous sodium hydroxide | 0.35 |
| Trehalose | 0.50 |
| N-acetyl glucosamine | 1.00 |
| Water/purified aribodopsis thaliana extract/lecithin | 0.50 |
| Aqueous solution acetyl hexapeptide-8 | 1.00 |
| Yeast ferment extract | 1.00 |
| Water/lecithin/micrococcus lysate | 0.50 |
| Milk protein/lactose/glucose/fructose | 0.50 |
| Saccharide isomerate | 0.50 |
| Whey protein | 0.50 |
| Water/butylene glycol/lecithin/lauryldimonium hydroxypropyl | 1.00 |
| hydrolyzed soy protein/lecithin/xanthan gum/ascorbyl | |
| tocopheryl maleate | |
| Glycerin/padina povonica extract | 0.10 |
| Thermus thermophillus ferment/glycerin | 0.05 |
| Camelina sativa seed oil | 0.05 |
| Water/gold/hydrolyzed wheat protein | 0.001 |
| Sorbitol/water/ascophyllum nodosum extract/ | 0.25 |
| asparagopsis armata extract | |
| Butylene glycol | 0.50 |
| Boswellia serrata extract | 0.05 |
| Calophyllum inophyllum (tamanu) seed oil | 0.05 |
| Fragrance | 0.20 |
| FD&C yellow No. 5 (1% aqueous solution) | 0.05 |
| Sodium acrylate/sodium acryloyldimethyl taurate | 1.00 |
| copolymer/hydrogenated polydecene/laureth-8 | |
| Trisodium resveratrol triphosphate | 0.50 |
| ROXISOMES ™ (water/Arabidopsis thaliana extract/lecithin) | 0.10 |
| ULTRASOMES-V ™ (water/lecithin/Micrococcus lysate) | 0.20 |

FORMULA 3

| Component | Wt % |
|---|---|
| Deionized water | QS |
| Hyaluronic acid | 9.00 |
| Caprylyl glycol | 0.28 |
| Dimethicone, 6 cst | 5.00 |
| Dimethicone (silicone gum/20 cst dimethicone blend) | 8.00 |
| Dimethicone/polysilicone 11 | 6.00 |
| Dimethicone/dimethicone PEG-10/15 crosspolymer | 1.00 |
| Lauryl PEG-9 polydimethylsiloxyethyl dimethicone | 1.00 |
| Wheat bran extract/olive extract | 0.20 |
| Lauryl PCA | 1.00 |
| Phenoxyethanol | 0.60 |
| Hexylene glycol | 0.05 |
| Mica/titanium dioxide | 0.75 |
| Mica/titanium dioxide/triethoxycaprylyl silane | 0.50 |
| N-acetyl glucosamine | 1.00 |
| Water/purified aribodopsis thaliana extract/lecithin | 1.00 |
| Aqueous solution acetyl hexapeptide-8 | 1.00 |
| Yeast ferment extract | 1.00 |
| Water/lecithin/micrococcus lysate | 0.50 |
| Milk protein/lactose/glucose/fructose | 0.50 |
| Whey protein | 0.560 |
| Water/butylene glycol/lecithin/lauryldimonium hydroxypropyl | 1.00 |
| hydrolyzed soy protein/lecithin/xanthan gum/ascorbyl | |
| tocopheryl maleate | |
| Glycerin/padina povonica extract | 0.10 |
| Aminomethyl propanol | 0.03 |
| Sodim phosphate dibasic (10% aqueous solution) | 0.75 |
| Citric acid (10% aqueous solution) | 0.008 |
| Sodium acrylate/sodium acryloyldimethyl taurate | 1.00 |
| copolymer/hydrogenated polydecene/laureth-8 | |
| Ammonium acrylodimethyltaurate/VP copolymer | 0.70 |
| Water/butylene glycol/decarboxy carnosine HCl | 0.50 |
| Trisodium resveratrol triphosphate | 0.50 |
| ROXISOMES ™ (waler/Arabidopsis thaliana extract/lecithin) | 0.10 |
| ULTRASOMES-V ™ (water/lecithin/Micrococcus lysate) | 0.20 |

While some illustrative embodiments of the inventions have been described hereinabove, such illustrative embodiments should not be interpreted in any manner to limit the broad scope of the prevent invention. Various modifications and equivalents of the described embodiments and components thereof will be apparent to those of ordinary skill in the art. Some modifications and equivalents will be readily recognized by one ordinarily skilled in the art, while others may require no more than routine experimentation. It is therefore understood that such modifications and equivalents are within the spirit and scope of the present invention.

What is claimed is:

1. A method for boosting or enhancing the natural repair responses in skin cells and improving cell survival rate against DNA damage caused by UVB radiation, comprising applying to skin, simultaneously or sequentially, resveratrol or a derivative thereof and at least one DNA repair enzyme.

2. The method of claim 1, wherein the resveratrol or a derivative thereof and the at least one DNA repair enzyme are contained in a single composition.

3. The method of claim 2, comprising sequentially treating the skin with at least two different compositions, in any order, wherein the first composition comprises the resveratrol or a derivative thereof and the at least one DNA repair enzyme, and wherein the second composition comprises at least one active ingredient that improves the efficacy of the first composition when both compositions are applied to skin.

4. The method of claim 1, wherein the resveratrol or a derivative thereof is contained in one composition and the at least one DNA repair enzyme is contained in a second composition and the compositions are applied to the skin sequentially in any order.

5. The method of claim 1, wherein the at least one DNA repair enzyme is selected from the group consisting of 8-oxoguanine DNA glucosylase, uracil-and-hypoxanthine-DNA-glycosylase, damaged-base glycosylase, 3-methyladenine-DNA-glycosylase, pyrimidine dimer-specific glycosylase, pyrimidine glycosylase/abasic lyase, N-glycosylase/apyrimidinic lyase, N-glycosylase/apurinic-apyrimidinic lyase, photolyase, O⁶-methylguanine-DNA-methyl transferase, T4 endonuclease V, pyrimidine dimer-specific endonuclease, apyrimidin/apurin-endonuclease, UV damage endonuclease, correndonuclease, and DNA-exonuclease.

6. The method of claim 5, wherein the at least one DNA repair enzyme comprises 8-oxoguanine DNA glucosylase, T4 endonuclease V, or a combination thereof.

7. The method of claim 1, wherein the topical composition comprises resveratrol.

8. The method of claim 1, wherein the topical composition comprises a derivative of resveratrol.

9. The method of claim 8, wherein the derivative of resveratrol is selected from the group consisting of resveratrol esters of inorganic acids, resveratrol esters of carboxylic acids, resveratrol ethers, N-substituted resveratrol, and combinations thereof.

10. The method of claim 9, wherein the resveratrol derivative is a resveratrol ester of a C₁₋₄₀ carboxylic acid.

11. The method of claim 10, wherein the resveratrol ester is selected from the group consisting of resveratrol butyrates, resveratrol valerates, resveratrol hexanoates, resveratrol sorbates, resveratrol laurates, resveratrol stearates, resveratrol palmitates, resveratrol oleates, resveratrol linoleates, resveratrol linolenates, resveratrol eicosapentaenoates, and resveratrol docosahexanoates.

12. The method of claim 10, wherein the resveratrol ester is selected from the group consisting of resveratrol ferulates.

13. The method of claim 9, wherein the resveratrol derivative is a resveratrol ester of an inorganic acid.

14. The method of claim 9, wherein the resveratrol ester is selected from the group consisting of resveratrol phosphates.

15. The method of claim 1, wherein the DNA repair enzyme or resveratrol or a derivative thereof, or both, are encapsulated by lipid vesicles.

16. The method of claim 1, wherein the topical composition is applied to the skin from about 1 to about 60 minutes prior to a period of bodily rest.

17. The method of claim 16, wherein the period of bodily rest is nightly sleep, a nap or meditation.

18. The method of claim 1, wherein the topical composition is a night cream, a night repair serum, or an overnight facial mask.

19. A skin care kit for boosting or enhancing the natural repair responses in skin cells and improving cell survival rate against DNA damage caused by UVB radiation, comprising a first receptacle containing a first composition comprising resveratrol or a derivative thereof and at least one DNA repair enzyme, and a second receptacle containing a second composition comprising at least one active ingredient that improves the efficacy of the first composition in the first receptacle when both compositions are applied to skin.

20. A skin care kit for boosting or enhancing the natural repair responses in skin cells and improving cell survival rate against DNA damage caused by UVB radiation, comprising a first receptacle containing a first composition comprising resveratrol or a derivative thereof, and a second receptacle containing a second composition comprising at least one DNA repair enzyme.

21. A topical composition for boosting or enhancing the natural repair responses in skin cells and improving cell survival rate against DNA damage caused by UVB radiation, comprising resveratrol or a derivative thereof and at least one DNA repair enzyme in a pharmaceutically or cosmetically acceptable vehicle.

22. The topical composition of claim 21, wherein the at least one DNA repair enzyme comprises 8-oxoguanine DNA glycosylate, T4 endonuclease V, or a combination thereof.

23. The topical composition of claim 15, wherein the derivative of resveratrol is selected from the group consisting of resveratrol esters of inorganic acids, resveratrol esters of carboxylic acids, resveratrol ethers, N-substituted resveratrol, and combinations thereof.

24. The topical composition of claim 23, wherein the resveratrol derivative is a resveratrol ester of a C₁₋₄₀ carboxylic acid.

25. The topical composition of claim 24, wherein the resveratrol ester is selected from the group consisting of resveratrol butyrates, resveratrol valerates, resveratrol hexanoates, resveratrol sorbates, resveratrol laurates, resveratrol stearates, resveratrol palmitates, resveratrol oleates, resveratrol linoleates, resveratrol linolenates, resveratrol eicosapentaenoates, and resveratrol docosahexanoates.

26. The topical composition of claim 24, wherein the resveratrol ester is selected from the group consisting of resveratrol ferulates.

27. The topical composition of claim 23, wherein the resveratrol derivative is a resveratrol ester of an inorganic acid.

28. The topical composition of claim 27, wherein the resveratrol ester is selected from the group consisting of resveratrol phosphates.

29. The topical composition of claim 21, wherein the DNA repair enzyme or resveratrol or a derivative thereof, or both, are encapsulated by lipid vesicles.
